(19) United States
(12) Patent Application Publication
(10) Pub. No.: US 2004/0265923 A1
(43) Pub. Date: Dec. 30, 2004

(54) Method and apparatus to determine the performance of protein arrays

(76) Inventors: James Gilmore, Woodlands, TX (US); Steven Daniel, Woodlands, TX (US); Mike Hogan, Conroe, TX (US); Sunny Tam, Missouri City, TX (US); Rick Wiese, The Woodlands, TX (US)

Correspondence Address: VINSON & ELKINS L.L.P., 1001 FANNIN STREET, 2300 FIRST CITY TOWER, HOUSTON, TX, 77002-6760, US

(21) Appl. No.: 10/476,742

(22) PCT Filed: May 3, 2002
(86) PCT No.: PCT/US02/13923

Related U.S. Application Data
(60) Provisional application No. 60/288,635, filed on May 3, 2001.

(51) Int. Cl. G01N 33/53; G01N 33/542

(52) U.S. Cl. 435/7.9

(57) ABSTRACT

The invention is directed to methods for measuring the performance of protein microarrays. The invention provides a multiplex micro-ELISA system.

This application claims the benefit of U.S. Provisional Application No. 60/288,635, filed May 3, 2001.

### TECHNICAL FIELD

This invention relates generally to cell biology, proteomics and polypeptide array, or “biochip,” technology. In particular, the invention is directed to methods for measuring the performance of microarrays.

### BACKGROUND

Protein microarrays often have immobilized capture antibodies. In many cases, the polypeptides are bound to glass or other treated surfaces, often through a biotin-streptavidin conjugation. The arrays are then incubated with solution containing antigen that will bind to the capture antibodies in a manner dependent upon time, buffer components, and recognition specificity. The antigens may then be visualized directly if they have been previously labeled, or may be allowed to bind to a secondary labeled antibody. The means of visualizing the amount of antigen bound to the capture antibody is dependent upon the labeling method utilized. In array formats, this is often by a CCD imager or laser scanner using filter sets that are appropriate to excite and detect the emissions of the label. The imager converts the amount of detected photons into an electronic signal (often an 8-bit or 16-bit scale) that can then be analyzed using software packages.

A major challenge to analyzing protein microarray data is determining the validity of the experimental data that has been generated, which can be in question if the results are due to errors in processing the arrays, or, if inappropriate amounts of sample were applied, or, if the cause of background values is not known or properly evaluated.

### SUMMARY

The invention provides methods for qualitatively and quantitatively analyzing a microarray's ability to detect and measure the amount of an analyte in a sample.

The invention provides a method for determining the performance of a protein or a small molecule array comprising the following steps: (a) providing a protein or a small molecule array comprising a plurality of biosites, each biosite comprising a plurality of polypeptide or small molecule capture probes immobilized to a substrate surface, wherein substantially all of the capture probes in a biosite have the same binding specificity for a target molecule, wherein at least one biosite comprises a capture probe capable of specifically binding to at least one control molecule; (b) providing a sample comprising a target molecule; (c) providing at least one control molecule, wherein at least one biosite of the array comprises a capture probe specific for the control molecule; (d) adding a known amount of the control molecule to the sample; (e) contacting the control molecule-added sample to the array and detecting to which biosite the target molecule and the control molecule have bound and the relative signal intensities of the bound target molecule and the bound control molecule on the biosite, thereby determining the performance of the array.

In one aspect of the methods of the invention, the sample is divided into at least two fractions and a known amount of a control molecule is added to one fraction. In another aspect, the sample is divided into at least two fractions and a known amount of a control molecule is added to each fraction. The sample can be divided into at least two fractions and a known amount of at least two different control molecules is added to each fraction or to different fractions. Each fraction can contain a control molecule at a different known amount of the control molecule, for example, serial dilutions of a control molecule can be designed.

In alternative aspects of the methods of the invention, the target molecule comprises a polypeptide, a lipid, a nucleic acid or a carbohydrate. The polypeptide capture probe can comprise a peptide or a peptidomimetic. The polypeptide capture probe can also comprise an antibody.

In one aspect, the array comprises biosites comprising at least two different antibodies (typically, only one type of capture molecule, e.g., antibody, per biosite) capable of specifically binding to the same target molecule, wherein each antibody binds to different epitopes on the target molecule. The array can comprise biosites comprising at least two different antibodies capable of specifically binding to the same target molecule, wherein each antibody binds to a same epitopes on the target molecule but with different affinity.

In alternative aspects of the methods of the invention, the control molecule comprises a polypeptide, a polysaccharide and a small molecule. The control molecule can comprise a detectable moiety. The detectable moiety can be selected from the group consisting of a radioactive moiety, a calorimetric moiety, a bioluminescent moiety, a fluorescent moiety and a chemiluminescent moiety.

In one aspect, the methods further comprise addition of a detection probe. The detection probe can be added at any step in the method, e.g., before, during or after step (e). The detection probe comprises any detectable moiety and the detection probe specifically binds to the control molecule or the target molecule. More than one detection probe can be added to one sample (to one fraction of a sample), e.g., one probe binding to the capture molecule, one binding to the target molecule, or both. The detectable moiety can be selected from the group consisting of a radioactive moiety, a calorimetric moiety, a bioluminescent moiety, a fluorescent moiety and a chemiluminescent moiety. The calorimetric moiety can be a dye, such as bromophenol blue. The method can further comprise addition of at least two detection probes, wherein a first detection probe specifically binds to the control molecule and a second detection probe specifically binds to the target molecule.

The detecting step can be performed by any device, or, be visual. In alternative aspects, the detecting step is performed by an optical or an electrical device (see below for complete discussion of detection devices).

In one aspect, the sample can be divided into at least two fractions and an amount of control molecule added to a first fraction is equivalent to a minimally detectable signal level for its binding to a biosite and an amount of control molecule added to a second fraction is equivalent to a saturated detectable signal level for its binding to a biosite. In this way a dynamic range can be measured.

In one aspect, the determination of the performance of the array comprises measurement of a background signal. The determination of the performance of the array can comprise a correlation of the dynamic range of the capture probe specific binding to the target molecule. The determination of the performance of the array can comprise a correlation of the specific binding of serial dilutions of control molecule to the array.

The determination of the performance of the array can comprise a correlation of the specific binding of the control molecule to the array, wherein the array comprises at least two biosites comprising varying known amounts of the same capture probe. The determination of the performance of the array can comprise a correlation of known array-bound signal intensities.

The invention can comprise a method for determining the performance of a protein or a small molecule array comprising the following steps: (a) providing a protein or a small molecule array comprising a plurality of biosites, each biosite comprising a plurality of polypeptide or small molecule capture probes immobilized to a substrate surface, wherein substantially all of the capture probes in a biosite have the same binding specificity for a target molecule in a biological sample, wherein at least one biosite comprises a capture probe capable of specifically binding to a target molecule in the biological sample and at least one biosite comprises a capture probe capable of specifically binding to at least one housekeeping biological molecule in the sample; (b) providing a biological sample comprising a target molecule and the housekeeping biological molecule; (c) contacting the sample to the array and detecting to which biosite the target molecule and the housekeeping biological molecule have bound and the relative signal intensities of the bound target molecule and the bound housekeeping biological molecule on the biosite, thereby determining the performance of the array.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits cited herein are hereby expressly incorporated by reference for all purposes.

### DESCRIPTION OF DRAWINGS

FIG. 1 schematically sets forth a map of an array used in the exemplary methods described in Example 1.

FIG. 2 is an illustration representing an array image demonstrating specificity and standard curves, as described in Example 1.

FIG. 3A is a linear regression equation derived as set forth in Example 1, below. FIG. 3B is an antigen concentration graph and standard curves from data derived from application of sample to an array, as described in detail in Example 1, below

Like reference symbols in the various drawings indicate like elements.

### DETAILED DESCRIPTION

The invention provides methods for measuring the performance, e.g., the analyte binding efficiency, of arrays, particularly protein or small molecule arrays. In one aspect, this is accomplished by adding a known amount of at least one “control” analyte to the array solution (“spiking the sample”) or to the array surface. The controls may be analyzed by software and compared to established performance thresholds to determine the performance of one or more microarrays. The analyte controls are also used to establish the amount of antigen in a sample. The controls can also determine errors or defects in the microarrays or the processing of the microarrays, and to determine the quality of the sample.

In one aspect, the methods are directed to qualitative and quantitative analysis of a microarray's ability to detect and measure the amount of an analyte in a sample. For example, one exemplary method provides a multiplex micro-ELISA system, as described in detail in Example 1, below. This exemplary method of the invention allows for savings of materials and time in the construction of standard curves and the analysis of samples compared to traditional ELISA due to the fact that the standard curves can be run simultaneously. Another advantage of this ELISA system is the fact that the loss of a single data point (probe value) does not negate the value of a test well. This is due to redundancy (capture antibodies printed in duplicate) and the use of several capture antibody concentrations. The use of regression equations formed from the titration of capture antibody has a balancing effect on occasional outlying data points without over or under emphasizing their impact on the total set as well. In one exemplary format of this assay, a standard 96 well glass slide array is utilized. This format is easily assimilated to automation. Genotyping and gene expression can be readily automated, allowing for a further increase in knowledge gained per unit time and resources spent. This rapid, high-throughput format can be used with proteomics analyses.

In alternative aspects, the methods of the invention use microarrays comprising capture molecules that are antibodies, antigens, or antigens bound to a capture antibody, for specific binding to a target analyte molecule or a control molecule.

Measurements of target analyte molecule or a control molecule binding to biosites can use the signal intensity of bound detection probes, which can include labeled antigens, labeled capture antibodies, labeled antigens bound to un-labeled capture antibody, and the like.

In determining the performance quality of an array, a “control molecule” is added to the sample. The “control molecule” can be an antigen. The “control molecule” can be exogenous to the sample, or, can be an isolated or recombinant preparation of the “target molecule” to be detected and measured by the array. The exogenous molecule, e.g., polypeptide, e.g., antigen, can be derived or isolated from a different specie than the that from which the sample was derived. The exogenous molecule may be an exogenous antigen, e.g., a recombinant protein.

The “control molecule” (e.g., exogenous antigen) is added to the sample in known amounts, e.g., in a known concentration. Where there may be one or more “control molecules” (e.g., exogenous antigens) and where the “control molecule” are at different concentrations, a dynamic range (see definitions) of binding of the “control molecule” to biosites on the array can be measured. The dynamic range can be determined by use of exogenous antigens. This measurement is useful when amounts of proteins endogenous to the sample that are known or suspected to be at high and low concentrations are being detected and quantified.

The methods of the invention also provide a measurement of “specificity” of binding of analyte (e.g., “target molecules” in a sample) to capture probes on the array. The specificity can be determined by adding (or “spiking”) the sample with one or more “control molecules” (such as known antigens). The specificity also can be determined by using control molecules and/or capture antibodies of varying amino acid homologies (e.g., as compared to a desired target polypeptide molecule). In this aspect of the method, the specificity is determined by measuring and comparing the amount of binding of control analytes (e.g., antigens) with various capture molecules (e.g., antibodies) of varying amino acid homologies. The exogenous antigens can be of varying amino acid homologies as compared to each other or known or suspected target molecules endogenous to the sample to be tested. Sensitivity can be determined by measuring the binding of proteins endogenous to the sample that are known or suspected to be in low concentrations to the array. The “control molecules” (such as known antigens, e.g., exogenous antigen) can be added to sample or other fractions such that a dilution series is prepared. This is added to the array and binding to array is quantified.

In another aspect, “gross measurement” of target molecule (e.g., antigen) present in the solution is determined by measuring the amount of “housekeeper” antigens (e.g., polypeptides) present in the sample solution. In another aspect, the method includes a measurement of the degradation of antigen in the sample. The degradation can be determined by the difference in array-bound signal for one antigen between two or more capture antibodies specific for different epitopes (i.e., capture antibodies can recognize different regions of an antigen). For example, one capture antibody can be specific to a domain of the antigen that is more easily degraded than other domains, or degraded or modified under certain physiologic conditions (e.g., cell cycle conditions, metabolic conditions, and the like). In one aspect, the measurement of degradation is determined by use of a labeled antigen that is bound capture antibody (e.g., a metabolically labeled polypeptide). In one aspect, the degradation of labeled target molecule is determined by the loss of label.

In one aspect, a measurement of the amount of capture molecule (e.g., antibody) present on the array (e.g., present on each biosite) can be determined using labeled (directly or indirectly labeled) target molecule. The measurement can be determined by the amount of signal from each biosite (from the amount of labeled molecule). In one aspect, an antigen (e.g., a polypeptide) is bound to the array and it is desired to measure the amount of antigen on each biosite. In this example, a labeled antibody specific for the antigen can be used; its binding to the array is quantified.

In one aspect, one or more capture antibodies recognize different domains found in an antigen (e.g., an exogenous antigen added to a sample). The amount of antigen can be measured by use of a dye, such as bromophenol blue. In one aspect, a capture molecule is an antibody that is derived from a source (e.g., a specie) different from that of the sample.

In one aspect, the methods of the invention include use of markers that can determine the alignment of biosites on the array. A marker can be a labeled antibody or a labeled antigen, or a labeled antigen bound to capture antibody that can bind to a capture molecule of a biosite. In one aspect, the methods include a measurement of background signal. This measurement occurs in an area not containing a biosite (e.g., a capture antibody).

In one aspect, the methods of the invention include the correlation of bound target molecule (e.g., antigen) signal to a dilution series of antigen, labeled antigen, capture antibody, labeled capture antibody, or labeled antigen bound to a biosite capture molecule (e.g., an antibody). The methods can include a correlation of dynamic range, sensitivity, specificity, gross measurement of antigen, degradation of antigen, degradation of capture antibody, amount of capture antibody on the array, markers, and/or background, or any combination thereof. Measured parameters (including dynamic range, sensitivity, specificity, gross measurement of antigen, degradation of antigen, degradation of capture antibody, amount of capture antibody on the array, markers, and/or background) can be further correlated to a set of pre-defined signal intensities such that a rating or score is given to the array based on the correlation.

Definitions

Unless defined otherwise, all technical and scientific terms used herein have the meaning commonly understood by a person skilled in the art to which this invention belongs. As used herein, the following terms have the meanings ascribed to them unless specified otherwise.

The terms “array” or “microarray” or “protein array” or “proteome array” or “biochip” as used herein are used interchangeably herein, and include all known variations of these devices, as discussed in detail, below.

By “biosite” is meant the biological molecules or capture probes that are deposited on the top surface of a reaction substrate, or base material, of an array. Under appropriate conditions, an association, e.g., a specific binding, or hybridization, can occur between the probe and a target molecule. The components of the biological molecule form the biosite since there is the potential of an interaction or a reaction occurring between each component strand of the biological molecule and the target molecule. The maximum number of biosites per array will depend on the size of the array, or reaction vessel within an array, may vary, depending on the probe deposition technology (e.g., printing), the nature of the probe, the means used to assess binding and/or to determine the volume or shape of a biosite (for qiality control). For example, the size of a biosite on an array may depend on the practical optical resolution of the accompanying detector/imager. For example, an array of 16 (4×4 array) biosites may be deposited on the hybridization substrate or base material that eventually forms the bottom of the entire reaction vessel. In this example, each biosite may comprise a circle of approximately about 25 to 200 microns (μm) in diameter. Thus, for a 16 biosite array, each of the 16×200 μm diameter area contains a uniform field of probes attached to the hybridization substrate (base material) in a concentration which is highly dependent on the probe size and the well size. Each 25 to 200 μm diameter area can contain millions of probe molecules. Also, each of the 16 different biosites (probe sites) can contain one type of probe. Thus, 16 different probe types can be assayed in an array containing 16 biosites (4×4 array) per reaction chamber. As another example, four separate 10×10 arrays (400 biosites) can be generated to fit into one well of a 96 well microtiter plate with sufficient spacing between each of the 400 biosites. For this 10×10 format, 400 hybridization experiments are possible within a single reaction chamber corresponding to 38,400 (96×400) assays/hybridization that can be performed nearly simultaneously.

By “substrate” is meant the substrate that the biosites, or probes, are deposited on. “Substrates” can be selected from a variety of materials, without limitation, e.g., polyvinyl, polystyrene, polypropylene, polyester, vinyl, other plastics, glass, SiO₂, other silanes, nylon membrane, gold or platinum, see further examples described, below. The solid surfaces can be derivatized, e.g., thiol-derivatized biopolymers and organic thiols can be bound to a metal solid substrate; see, e.g., U.S. Pat. No. 5,942,397 (see below for more examples).

The term “immobilized” means that the probe can be attached to a surface (e.g., the substrate) in any manner or any method; including, e.g., reversible or non-reversible binding, covalent or non-covalent attachment, and the like.

The term “control molecule” means any molecule that is added in known amounts to the sample in the methods of the invention. The array is designed to comprise at least one biosite that specifically binds to each control molecule. The array can also be designed to have several biosites that bind to the same control molecule, but with different affinities.

The term “detection probe” means any molecule that can be directly or indirectly detected by any means, including electronic or visual methods; thus, the detection probe can comprise two molecules, including a first molecule (e.g., one that specifically binds the target molecule or the control molecule) and a second molecule that binds the first molecule. In one embodiment, the detection probe is a detectable moiety that comprises the target molecule or control molecule, e.g., the target or control molecule is a polypeptide phosphorylated with radioactive P³².

The term “dynamic range” means the difference between the most and least sensitive signal. For example, in one aspect, the sample is divided into at least two fractions and an amount of control molecule added to a first fraction is equivalent to a minimally detectable signal level for its binding to a biosite and an amount of control molecule added to a second fraction is equivalent to a saturated detectable signal level for its binding to a biosite; the difference between the minimally detectable and the saturated signal is a dynamic range.

The term “specificity” means the ability of a molecule (e.g., a protein or small molecule) to recognize and differentiate a second molecule (by “specifically binding to the second molecule). The term “sensitivity” means the minimum signal that can be recognized above background signal.

The term “background” means the signal generated by noise and/or non-specific binding. For example, background can be determined where a capture antibody has not been printed onto an array.

The term “degradation” means the loss of structural conformation in a protein, as for example through a deletion or alteration in the amino acid sequence. The term “markers” means capture antibodies that are printed in a pattern such that the orientation can be easily recognized.

The term “housekeepers” means antigens present in sample that are believed to vary little in concentration or composition from sample to sample. For example, housekeeping polypeptides are well known in the art, see, e.g., U.S. Pat. Nos. 5,876,978; 5,876,937.

The term “solution” means a liquid or semi-liquid that is comprised of varying buffers and/or samples and is applied to the array.

The term “antibody” refers to a peptide or polypeptide substantially encoded by an immunoglobulin gene or immunoglobulin genes, or fragments or equivalents thereof, capable of specifically binding an epitope, see, e.g. Fundamental Immunology, Third Edition, W. E. Paul, ed., Raven Press, N.Y. (1993); Wilson (1994) J. Immunol. Methods 175:267-73; Yarmush (1992) J. Biochem. Biophys. Methods 25:85-97. One of skill will appreciate that antibody fragments may be isolated or synthesized de novo either chemically or by utilizing recombinant DNA methodology. The term antibody also includes “chimeric” antibodies either produced by the modification of whole antibodies or those synthesized de novo using recombinant DNA methodologies. Typically, such chimeric antibodies are “humanized antibodies,” i.e., where the epitope binding site is generated from an immunized mammal, such as a mouse, and the structural framework is human. Immunoglobulins can also be generated using phage display libraries, and variations thereof Antibodies or other molecules that bind to post-translationally modified polypeptides are well known in the art, see, e.g., U.S. Pat. No. 6,008,024; 5,763,198; 5,599,681; 5,580,742. Methods of producing polyclonal and monoclonal antibodies are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif.; Goding, MONOCLONAL ANTIBODIES: PRINCIPLES AND PRACTICE (2d ed.) Academic Press, New York, N.Y. (1986); Harlow (1988) ANTIBODIES, A LABORATORY MANUAL, Cold Spring Harbor Publications, New York.

The term “small molecule” means any synthetic small molecule, such as an organic molecule or a synthetic molecule, such as those generated by combinatorial chemistry methodologies. These small molecules can be synthesized using a variety of procedures and methodologies, which are well described in the scientific and patent literature, e.g., Organic Syntheses Collective Volumes, Gilman et al. (Eds) John Wiley & Sons, Inc., NY; Venuti (1989) Pharm Res. 6:867-873. Synthesis of small molecules, as with all other procedures associated with this invention, can be practiced in conjunction with any method or protocol known in the art. For example, preparation and screening of combinatorial chemical libraries are well known to those of skill in the art, see, e.g., U.S. Pat. Nos. 6,096,496; 6,075,166; 6,054,047; 6,004,617; 5,985,356; 5,980,839; 5,917,185; 5,767,238.

Nucleic Acid and Polypeptide Probes

This invention provides an array comprising immobilized capture molecules, which can be immobilized polypeptides, nucleic acids or oligonucleotides (and polysaccharides, lipids or small molecules). The “target molecules” and “control molecules” can also be polypeptides, nucleic acids or oligonucleotides (and polysaccharides or small molecules). For example, a polypeptide can be immobilized to an array substrate surface by conjugation to an oligonucleotide, which in turn specifically hybridizes to a nucleic acid immobilized on the array surface (see, e.g., U.S. Pat. No. 6,083,763). These probes can be made and expressed in vitro or in vivo, any means of making and expressing polypeptides or nucleic acids used in the devices or practiced with the methods of the invention can be used. The invention can be practiced in conjunction with any method or protocol known in the art, which are well described in the scientific and patent literature.

The nucleic acids of the invention, whether, e.g., RNA, cDNA, fragments of genomic DNA, can be isolated from a variety of sources, genetically engineered, amplified, and/or expressed recombinantly (the polypeptides used in the invention can be recombinantly generated and/or genetically modified). Any recombinant expression system can be used, including, in addition to mammalian cells, e.g., bacterial, yeast, insect or plant systems. Alternatively, these nucleic acids can be synthesized in vitro by well-known chemical synthesis techniques, as described in, e.g., Carruthers (1982) Cold Spring Harbor Symp. Quant. Biol. 47:411-418; Belousov (1997) Nucleic Acids Res. 25:3440-3444; Frenkel (1995) Free Radic. Biol. Med. 19:373-380; Blommers (1994) Biochemistry 33:7886-7896; Beaucage (1981) Tetra. Lett. 22:1859; U.S. Pat. No. 4,458,066.

Techniques for the manipulation of nucleic acids and generating recombinant polypeptide, such as, e.g., generating mutations in sequences, subcloning, labeling probes, sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Capture molecules, control molecules and detection probes can include, e.g., amino acids, peptides, oligopeptide, polypeptides, peptidomimetics, other short polymers or organic molecules. When amino acids are used, alternative embodiment can use methyl esters because of commercial availability and the fact that they are not altered by the formation reactions (binding of the association surface to the support surface). “Peptidomimetics” include synthetic chemical compounds that have substantially the same structural and/or functional characteristics of the corresponding composition, e.g., the peptides, oligopeptides (e.g., oligo-histidine, oligo-aspartate, oligo-glutamate, poly-(his)₂(gly)₁, and poly-(his)₂(asp)₁), polypeptides, imidazole derivatives or equivalents used in the association surface of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropyl-carbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, p 267-357, Marcell Dekker, N.Y.).

Arrays, or “BioChips”

The invention provides methods for determining the performance (e.g., the binding efficiency) of arrays, particularly protein and small molecule arrays. Arrays used in the methods of the invention comprise a plurality of “capture probes,” each immobilized element comprising a defined amount of one or more molecules. The capture probes are immobilized onto a solid surface for binding (directly or indirectly) to a target molecule or a control molecule. The biosites may be arranged on the solid surface at different sizes and different densities. The methods of the invention can incorporate in whole or in part designs of arrays, and associated components and methods, as described, e.g., in U.S. Pat. Nos. 6,197,503; 6,174,684; 6,156,501; 6,093,370; 6,087,112; 6,087,103; 6,087,102; 6,083,697; 6,080,585; 6,054,270; 6,048,695; 6,045,996; 6,022,963; 6,013,440; 5,959,098; 5,856,174; 5,843,655; 5,837,832; 5,770,456; 5,723,320; 5,700,637; 5,695,940; 5,556,752; 5,143,854; see also, e.g., WO 99/51773; WO 99/09217; WO 97/46313; WO 96/17958; WO 89/10977; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32; Epstein (2000) Current Opinion in Biotech. 11:36-41; Mendoza (1999) “High-throughput microarray-based enzyme-linked immunosorbent assay (ELISA),” Biotechniques 27: 778-788; Lueking (1999) Protein microarrays for gene expression and antibody screening,” Anal. Biochem. 270:103-111; Davies (1999) “Profiling of amyloid beta peptide variants using SELDI protein chip arrays,” Biotechniques 27:1258-1261.

Probe Deposition onto Substrate

The invention provides for making an array by immobilizing onto a substrate a plurality of biosites comprising “capture probes.” The probes can be “deposited” or immobilized” onto the substrate using any method or combination of methods known in the art, e.g., pizo-electric, such as ink-jet, processes and systems, robotic deposition, photolithographic in-situ synthesis, use of microsyringes, or a continuous flow bundled microcapillary process (see, e.g., U.S. Pat. No. 6,083,763). Array fabrication methods that can be incorporated, in whole or in part, in the making or using of the invention include, e.g., those described in U.S. Pat. Nos. 6,197,503; 6,177,238; 6,164,850; 6,150,147; 6,083,763; 6,048,695; 6,010,616; 5,599,695; 5,919,523; 5,861,242; 5,770,722; 5,750,669; 5,143,854.

Substrate Surfaces

The arrays used in the methods of the invention can comprise substrate surfaces of a rigid, semi-rigid or flexible material. The substrate surface can be flat or planar, be shaped as wells, raised regions, etched trenches, pores, beads, filaments, or the like. Substrates can be of any material upon which a “capture probe” can be directly or indirectly bound. For example, suitable materials can include paper, glass (see, e.g., U.S. Pat. No. 5,843,767), ceramics, quartz or other crystalline substrates (e.g. gallium arsenide), metals, metalloids, polacryloylmorpholide, various plastics and plastic copolymers, Nylon™, Teflon™, polyethylene, polypropylene, poly(4-methylbutene), polystyrene, polystyrene/latex, polymethacrylate, poly(ethylene terephthalate), rayon, nylon, poly(vinyl butyrate), polyvinylidene difluoride (PVDF) (see, e.g., U.S. Pat. No. 6,024,872), silicones (see, e.g., U.S. Pat. No. 6,096,817), polyformaldehyde (see, e.g., U.S. Pat. Nos. 4,355,153; 4,652,613), cellulose (see, e.g., U.S. Pat. No. 5,068,269), cellulose acetate (see, e.g., U.S. Pat. No. 6,048,457), nitrocellulose, various membranes and gels (e.g., silica aerogels, see, e.g., U.S. Pat. No. 5,795,557), paramagnetic or superparamagnetic microparticles (see, e.g., U.S. Pat. No. 5,939,261) and the like. The substrate can be derivatized for application of other compounds upon which the probes are immobilized. Reactive functional groups can be, e.g., hydroxyl, carboxyl, amino groups or the like. Silane (e.g., mono- and dihydroxyalkylsilanes, aminoalkyltrialkoxy-silanes, 3-aminopropyl-triethoxysilane, 3-aminopropyltrimethoxysilane) can provide a hydroxyl functional group for reaction with an amine functional group.

Detection Probes and Devices

The detection probe can comprise any detectable moiety, including, e.g., radioactive, colorimetric, bioluminescent, fluorescent or chemiluminescent or another photon detectable moieties. The detection probe also comprises any molecule that specifically binds to the target molecule when the target molecule is specifically bound to the capture probe. The detection probe can comprise a polypeptide, a lipid, a small molecule, a polysaccharide, a nucleic acid or a combination thereof. “Detectable moieties,” such as fluorescent, bioluminescent or chemiluminescent, or radiation, can be detected and quantified, e.g., using assays and devices well known in the art, as described in, e.g., U.S. Pat. Nos. 6,225,670; 6,211,524; 6,198,835; 6,197,928; 6,197,499; 6,194,731; 6,194,223; 6,191,852; 6,191,425; 6,132,983; 6,087,476; 6,060,261; 5,866,348; 5,094,939; 5,744,320; 5,631,734; 5,192,980; 5,091,652.

The binding of the “detection probe” to the molecule to be analyzed can be performed in any manner using any detection device, e.g., by scanning the substrate surface and determining if any or sufficient detection probe has been bound to molecule affixed to a biosite on the substrate surface area. These functions can be performed by any device, e.g., an optical or an electrical device.

For example, an imaging system can be a proximal charge-coupled device (CCD) detection/imaging; due to its inherent versatility, it can also accommodate chemiluminescence, fluorescent and radioisotope target molecule detection, high throughput, and high sensitivity. This detection/imaging apparatus can include a lensless imaging array comprising a plurality of solid state imaging devices, such as an array of CCDs, photoconductor-on-MOS arrays, photoconductor-on-CMOS arrays, charge injection devices (CIDs), photoconductor on thin-film transistor arrays, amorphous silicon sensors, photodiode arrays, or the like.

The devices and methods of the invention incorporate in whole or in part designs of detection devices as described, e.g., in U.S. Pat. Nos. 6,197,503; 6,197,498; 6,150,147; 6,083,763; 6,066,448; 6,045,996; 6,025,601; 5,599,695; 5,981,956; 5,698,089; 5,578,832; 5,632,957.

### EXAMPLES

The following examples are offered to illustrate, but not to limit the claimed invention.

### Example 1

### Determining the Performance of an Array

Exemplary methods for practicing the methods of the invention are provided, including analysis of the performance of a protein array.

Materials and Methods

Slide Preparation. Standard 96 well glass slides (e.g., from Genometrix Genomics, Inc., The Woodlands, Tex.) were cleaned and silanized. Arrays were printed on prepared slides with a capillary printer (e.g., from Genometrix Genomics, Inc.). Print solutions consisted of appropriate monoclonal capture antibodies diluted no less than 1:1 in print buffer (0.1 M carbonate buffer, pH 9.5+5% glycerol). The anti-total PSA and PSA-ACT capture antibodies were purchased from Diagnostic Systems Laboratories (#A-160, Webster, Tex.) and Fitzgerald Industries International (#10-P22, Concord, Mass.), respectively. The anti-IL-6 capture antibody was purchased from Pharmingen (#26451E, San Diego, Calif.). The positional and positive control marker used for these arrays was rabbit IgG (Fitzgerald, #31-RGGO) used at a print concentration of 150 μg/ml. The slides were visually inspected after printing for quality of print.

MicroELISA assay. After overnight storage at 4° C., the sample wells were first rinsed in triplicate then blocked on a shaker plate for one hour at room temperature in a blocker casein (#37528ZZ, Pierce Chemical Co., Rockford, Ill.). Blocker casein was aspirated from the wells and appropriate antigen solutions, diluted in PBS, were added to each of the test wells. Antigen proteins and suppliers were as follows: PSA and PSA-ACT, Fitzgerald Industries (#30-AP16 and 30-AP13, respectively), recombinant human IL-6, Pharmingen (#26456E). The array plate was placed in a humidity chamber and incubated at 37° C. for 2 hours. After sample incubation, the plate was removed from the oven and washed 3 times with blocker casein. Detection antibodies were next applied to every well. Detection antibodies were rabbit anti-PSA and anti-IL-6 polyclonal antibodies, both purchased from Fitzgerald Industries (#20-PR50 and 20IR-09, respectively). The samples were once again incubated at 37° C. in a humidity chamber, for an hour and a half An alkaline phosphatase-linked goat anti-rabbit secondary antibody (Pierce, #31342ZZ) was used to probe for the detection antibodies. After a one-hour, room temperature (RT) incubation, manufacturers instructions were followed to generate the enzyme linked fluorescence signal used to detect antigen binding (Molecular Probes, #E-6604, Eugene, Oreg.).

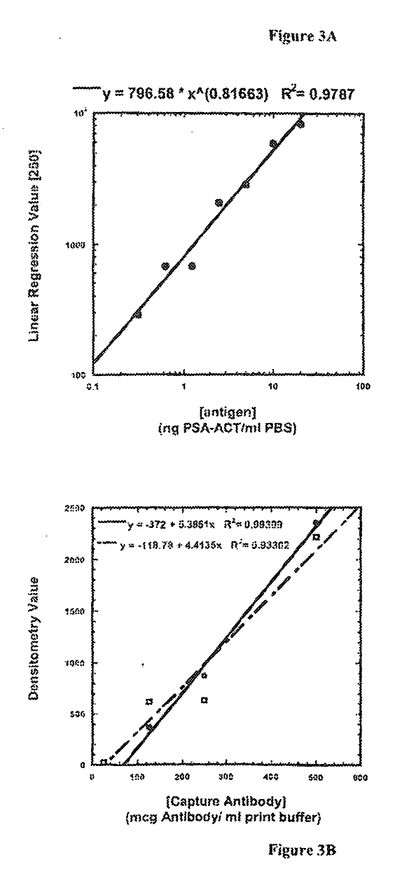

The completed assay slide was imaged utilizing a CCD camera controlled by software (Genometrix Genomics, Inc.). Varying exposure times were taken to allow for the imaging of subject proteins generating signals of significantly differing intensities. The saved TIFF images were finally analyzed utilizing dot scoring software that is designed to automatically subtract background from the utilized densitometry values. Dot score values were used to construct densitometry versus capture antibody concentration graphs for each individual well (antigen concentration) of the standard curve. The linear regression equations derived from each of these graphs were used to generate values corresponding to the densitometry value of the second highest capture antibody concentration for each well (250 μg/ml for the PSA antibodies and 125 μg/ml for the IL-6 antibody). Since the arrays were printed in duplicate this procedure was followed for each set of data from each well and the values obtained were averaged. As an example, in FIG. 3A, the two linear regression equations derived for the two data sets plotted are shown, for this data, the X value of 250 μg/ml would be substituted into each of the regression equations and the obtained y values averaged to yield the linear regression value used on the standard curve graph at this particular antigen concentration. This process is repeated for each substrate at each concentration in each of the standard curves.

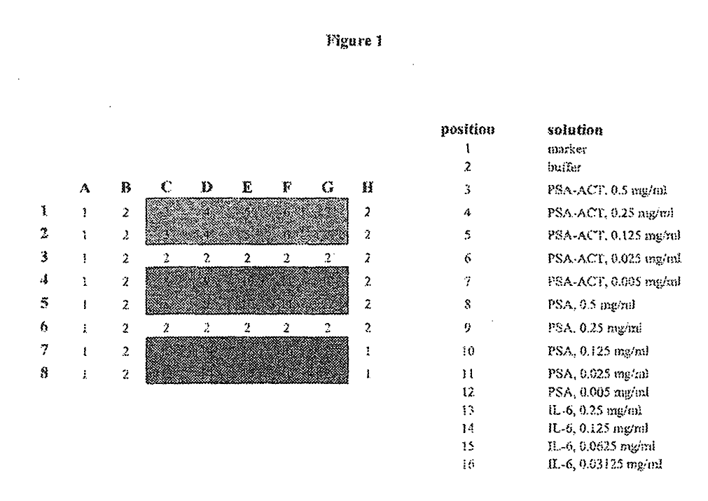

The array used for these experiments is configured as an 8×8 array of printed antibody, one array per well in a standard 8×12 microtiter format; the specific array design is illustrated in FIG. 1. The 64-element array contained a 5 element dilution series in duplicate for both forms of PSA and a 4 element dilution series printed in duplicate for IL-6. The rabbit IgG markers printed in positions A1-A8 and H-7 and 8 are useful for the orientation and identification of probes within the array.

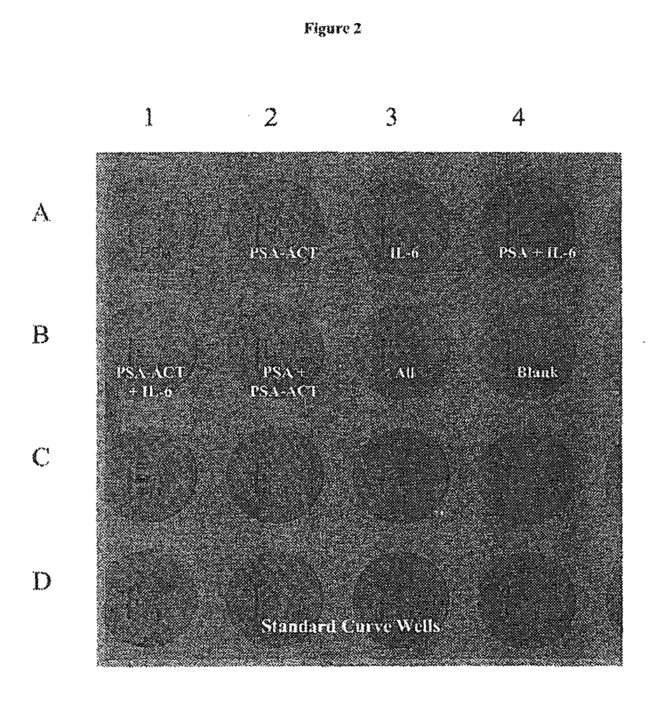

FIG. 2 is an image of 16 wells, which demonstrates the selectivity of the antibodies for the appropriate antigen (A1-B3), and contains the 7-point standard curve assayed in tandem for the 3 proteins of interest (C1-D3). Wells B4 and D4 are both negative controls (no recombinant protein added). As expected, in well A1 (PSA only) signal is detected only at the total PSA capture probes, in A2 (PSA-ACT) signal is detectable at both the total PSA and specific ACT bound PSA capture probes, and in A3 (IL-6) detectable signal emanates solely from the IL-6 probes. Additionally, for each of the combinations of these substrates only those probes specific for the added antigen yield a detectable signal (A4-B3). Densitometry values obtained from the standard curve wells (C1-D3) were used to construct a graph of densitometry value versus capture (printed) antibody concentration for each of the 3 antigens examined in each well for each antigen concentration, an example of one of these graphs is shown in FIG. 3A. The linear regression equations derived from each of these graphs were then used to derive the points for the linear regression value versus antigen concentration graph (standard curves) shown in FIG. 3B. For the total PSA curve the highest concentration is omitted so upper limits will match on both PSA forms (PSA total concentration is sum of PSA and PSA-ACT so the titration curve for detectable antigen actually covers the range 40 ng/ml to 0.625 ng/ml for the total PSA antibody). The correlation coefficients derived from the regression lines are comparable, if not superior, to those attained utilizing standard ELISA.

This multiplex micro-ELISA system of the invention allows for savings of materials and time in the construction of standard curves and the analysis of samples compared to traditional ELISA due to the fact that the standard curves can be run simultaneously (all analytes in a single well) instead of single or replicate wells for each concentration of each antigen or sample. In addition, to time and sample savings (only 25 μl of sample is needed), capture antibody usage is decreased in this system as well. As an example, 40 μl of the IL-6 capture antibody would be necessary to prepare one 96 well microtiter plate for standard ELISA according to the manufacturers recommended dilutions. Performing protein quantification by the microELISA methods of the invention, and utilizing array construction by capillary printer, it is possible to print more than a hundred 96 well arrays with this same 40 μl of capture antibody.

The information available from each well is significantly greater in this microarray configuration as compared to a standard ELISA as well. In standard ELISA, the values used to determine analyte concentration are 3 sample absorbance values (if the test is performed in triplicate), here the number of data points used to determine these concentrations are often twice that number and no less then equal to it at the lower analyte concentrations, utilizing a single well and multiple antibody dilutions printed in duplicate. The use of a capture antibody dilution series allows for a greater working range in the invention's ELISA format as well. As the antigen concentration increases lower capture antibody concentration probes are detectable, and as the higher detection probe concentrations become saturated the lower probe concentrations can be used for quantification. This factor virtually eliminates the necessity of having to dilute samples and repeat an assay; this is especially valuable when working with limited sample amounts. As an example of this, the PSA (total) array is capable of detecting PSA at concentrations up to 100 ng/ml. Additionally, this array design is not constrained by the need to analyze proteins present within the sample at approximately equal concentrations. In the experiments reported herein, there is approximately a 500-fold difference in protein concentrations from highest (PSA, 20 ng/nl) to lowest (IL-6, 0.0046875 ng/ml), other work we have completed has demonstrated a range of approximately 400,000-fold (2 mg/ml to 4 pg/ml).

The microarray ELISA method of the invention is expandable to the standard array size (16×16 elements) used in typical production procedures (e.g., Genometrix Genomics, Inc.), which would allow for the determination of 20 to 30 individual proteins within a single array. Polyclonal antibodies were used as detector antibodies in this array and no cross reactivity was detected, therefore, it would be hypothesized that larger arrays made entirely of monoclonal antibodies should have no problem with cross-reactivity as well (possibly polyclonal detector antibodies will not encounter problems at greater densities either, so long as monoclonal capture antibodies are utilized exclusively).

A number of embodiments of the invention have been described. Nevertheless, it will be understood that various modifications may be made without departing from the spirit and scope of the invention. Accordingly, other embodiments are within the scope of the following claims.

What is claimed is:

1. A method for determining the performance of a protein or a small molecule array comprising the following steps:
(a) providing a protein or small molecule array comprising a plurality of biosites, each biosite comprising a plurality of polypeptide or small molecule capture probes immobilized to a substrate surface, wherein substantially all of the capture probes in a biosite have the same binding specificity for a target molecule, wherein at least one biosite comprises a capture probe capable of specifically binding to at least one control molecule;
(b) providing a sample comprising a target molecule;
(c) providing at least one control molecule, wherein at least one biosite of the array comprises a capture probe specific for the control molecule;
(d) adding a known amount of the control molecule to the sample; and
(e) contacting the control molecule-added sample to the array and detecting to which biosite the target molecule and the control molecule have bound and the relative signal intensities of the bound target molecule and the bound control molecule on the biosite, thereby determining the performance of the array.

2. The method of claim 1, wherein the sample is divided into at least two fractions and a known amount of a control molecule is added to one fraction.

3. The method of claim 1, wherein the sample is divided into at least two fractions and a known amount of a control molecule is added to each fraction.

4. The method of claim 1, wherein the sample is divided into at least two fractions and a known amount of at least two different control molecules is added to each fraction or to different fractions.

5. The method of claim 3 or claim 4, wherein each fraction containing a control molecule has a different known amount of the control molecule.

6. The method of claim 1, wherein the target molecule comprises a polypeptide.

7. The method of claim 1, wherein the target molecule is selected from the group consisting of a lipid, a nucleic acid and a carbohydrate.

8. The method of claim 1, wherein the polypeptide capture probe comprises a peptide.

9. The method of claim 1, wherein the polypeptide capture probe comprises an antibody.

10. The method of claim 9, wherein the array comprises biosites comprising at least two different antibodies capable of binding to the same target molecule, wherein each antibody binds to a different epitope on the target molecule.

11. The method of claim 9, wherein the array comprises biosites comprising at least two different antibodies capable of binding to the same target molecule, wherein each antibody binds to a same epitope on the target molecule but with different affinities.

12. The method of claim 1, wherein the control molecule comprises a polypeptide.

13. The method of claim 1, wherein the control molecule comprises a polysaccharide.

14. The method of claim 1, wherein the control molecule comprises a small molecule.

15. The method of claim 1, wherein the control molecule comprises a detectable moiety.

16. The method of claim 15, wherein the detectable moiety is selected from the group consisting of a radioactive moiety, a colorimetric moiety, a bioluminescent moiety, a fluorescent moiety and a chemiluminescent moiety.

17. The method of claim 1, further comprising the addition of a detection probe, wherein the detection probe comprises a detectable moiety and the detection probe specifically binds to the control molecule or the target molecule.

18. The method of claim 17, wherein the detectable moiety is selected from the group consisting of a radioactive moiety, a colorimetric moiety, a bioluminescent moiety, a fluorescent moiety and a chemiluminescent moiety.

19. The method of claim 18, wherein the colorimetric moiety is a dye.

20. The method of claim 18, wherein the dye is a bromophenol blue.

21. The method of claim 17, further comprising addition of at least two detection probes, wherein a first detection probe specifically binds to the control molecule and a second detection probe specifically binds to the target molecule.

22. The method of claim 1, wherein the detecting of step (e) is performed by an optical or electrical device.

23. The method of claim 1, wherein the sample is divided into at least two fractions and an amount of control molecule added to a first fraction is equivalent to a minimally detectable signal level for its binding to a biosite and an amount of control molecule added to a second fraction is equivalent to a saturated detectable signal level for its binding to a biosite.

24. The method of claim 1, wherein determination of the performance of the array comprises measurement of a background signal.

25. The method of claim 1, wherein determination of the performance of the array comprises a correlation of the dynamic range of the capture probe specific binding to the target molecule.

26. The method of claim 1, wherein determining the performance of the array comprises correlating the specific binding of serial dilutions of control molecule to the array.

27. The method of claim 1, wherein determining the performance of the array comprises correlating the specific binding of the control molecule to the array, wherein the array comprises at least two biosites comprising varying known amounts of the same capture probe.

28. The method of claim 1, wherein determining the performance of the array comprises correlating known array-bound signal intensities.

29. A method for determining the performance of a protein or a small molecule array comprising the following steps:
(a) providing a protein or small molecule array comprising a plurality of biosites, each biosite comprising a plurality of polypeptide or small molecule capture probes immobilized to a substrate surface,
wherein at least one biosite comprises a capture probe capable of binding to a target molecule in the biological sample and at least one biosite comprises a capture probe capable of binding to at least one housekeeping biological molecule in the sample;
(b) providing a biological sample comprising a target molecule and the housekeeping biological molecule;
(c) contacting the sample to the array and detecting to which biosite the target molecule and the housekeeping biological molecule have bound and the relative signal intensities of the bound target molecule and the bound housekeeping biological molecule on the biosite.

30. A method for determining the performance of a protein or small molecule array comprising the following steps:
(a) providing a protein or small molecule array having a plurality of biosites, wherein at least one biosite includes a capture probe capable of binding to at least one control molecule;
(b) providing a sample, wherein the sample includes a target molecule;
(c) providing at least one control molecule, wherein at least one biosite of the array includes a capture probe capable of binding to the control molecule;
(d) adding a known amount of the control molecule to at least a portion of the sample;
(e) contacting the control molecule-added sample to the array;
(f) detecting which biosites include the target molecule and the control molecule; and
(g) determining the relative signal intensities of the target molecule and the control molecule on biosites including the target molecule and the control molecule.

31. The method of claim 30, wherein the sample is divided into at least two fractions and a known amount of a control molecule is added to at least one fraction.

32. The method of claim 30, wherein the sample is divided into at least two fractions and a known amount of a control molecule is added to each fraction.

33. The method of claim 30, wherein the sample is divided into at least two fractions and a known amount of at least two different control molecules is added to at least one fraction.

34. The method of claim 33, wherein each fraction containing a control molecule has a different known amount of the control molecule.

35. The method of claim 30, wherein the sample is divided into at least two fractions and a known amount of at least two different control molecules is added to each fraction.

36. The method of claim 35, wherein each fraction containing a control molecule has a different known amount of the control molecule.

37. The method of claim 30, wherein the sample is divided into at least two fractions and a known amount of at least two different control molecules is added to different fractions.

38. The method of claim 37, wherein each fraction containing a control molecule has a different known amount of the control molecule.

39. The method of claim 30, wherein the target molecule comprises a polypeptide.

40. The method of claim 30, wherein the target molecule is selected from the group consisting of a lipid, a nucleic acid, and a carbohydrate.

41. The method of claim 30, wherein the capture probe includes a peptide.

42. The method of claim 30, wherein the capture probe includes an antibody.

43. The method of claim 42, wherein the array includes biosites having at least two different antibodies capable of binding to the same target molecule, wherein each antibody binds to a different epitope on the target molecule.

44. The method of claim 9, wherein the array includes biosites having at least two different antibodies capable of binding to the same target molecule, wherein each antibody binds to a same epitope on the target molecule but with different affinities.

45. The method of claim 30, wherein the capture probe includes a small molecule.

46. The method of claim 30, wherein the control molecule includes a polypeptide.

47. The method of claim 30, wherein the control molecule includes a polysaecharide.

48. The method of claim 30, wherein the control molecule includes a small molecule.

49. The method of claim 30, wherein the control molecule includes a detectable moiety.

50. The method of claim 49, wherein the detectable moiety is selected from the group consisting of a radioactive moiety, a colorimetric moiety, a bioluminescent moiety, a fluorescent moiety, and a chemiluminescent moiety.

51. The method of claim 30, further comprising the addition of a detection probe, wherein the detection probe includes a detectable moiety and the detection probe binds to the control molecule.

52. The method of claim 51, wherein the detectable moiety is selected from the group consisting of a radioactive moiety, a colorimetric moiety, a bioluminescent moiety, a fluorescent moiety, and a chemiluminescent moiety.

53. The method of claim 52, wherein the colorimetric moiety is a dye.

54. The method of claim 52, wherein the dye is a bromophenol blue.

55. The method of claim 51, further comprising the addition of at least two detection probes, wherein a first detection probe specifically binds to the control molecule and a second detection probe specifically binds to the target molecule.

56. The method of claim 30, further comprising the addition of a detection probe, wherein the detection probe includes a detectable moiety and the detection probe binds to the target molecule.

57. The method of claim 56, wherein the detectable moiety is selected from the group consisting of a radioactive moiety, a colorimetric moiety, a bioluminescent moiety, a fluorescent moiety, and a chemiluminescent moiety.

58. The method of claim 57, wherein the colorimetric moiety is a dye.

59. The method of claim 57, wherein the dye is a bromophenol blue.

60. The method of claim 56, further comprising the addition of at least two detection probes, wherein a first detection probe specifically binds to the control molecule and a second detection probe specifically binds to the target molecule.

61. The method of claim 30, wherein the detecting of step (e) is performed by an optical or electrical device.

62. The method of claim 30, wherein the sample is divided into at least two fractions and an amount of control molecule added to a first fraction is equivalent to a minimally detectable signal level for its binding to a biosite and an amount of control molecule added to a second fraction is equivalent to a saturated detectable signal level for its binding to a biosite.

63. The method of claim 30, wherein a determination of the performance of the array includes measurement of a background signal.

64. The method of claim 30, wherein a determination of the performance of the array includes a correlation of the dynamic range of the capture probe binding to the target molecule.

65. The method of claim 30, wherein a determination of the performance of the array includes a correlation of the binding of serial dilutions of the control molecule to the array.

66. The method of claim 30, wherein a determination of the performance of the array comprises a correlation of the specific binding of the control molecule to the array, wherein the array comprises at least two biosites comprising varying known amounts of the same capture probe.

67. The method of claim 1, wherein a determination of the performance of the array includes a correlation of known array-bound signal intensities.

68. A method for determining the performance of a protein or a small molecule array comprising the following steps:
(a) providing a protein or small molecule array having a plurality of biosites, each biosite including a plurality of capture probes immobilized to a substrate surface, wherein at least one biosite includes a capture probe capable of binding to a target molecule, wherein at least one capture probe is capable of binding to at least one housekeeping molecule;
(b) providing a biological sample including a target molecule and the housekeeping molecule;
(c) contacting the sample to the substrate surface;
(d) detecting to which biosite the target molecule and the housekeeping molecule have bound; and
(e) determining the relative signal intensities of the bound target molecule and the bound housekeeping molecule on the biosite.
